SEQUENCE LISTING (6 sequences)

SEQ ID NO: 1 - DNA, 25 bp, Streptococcus suis
  misc_feature: Oligo Probe 1
tgttgacggcaacattgttgagtcc

SEQ ID NO: 2 - DNA, 27 bp, Streptococcus suis
  misc_feature: Oligo probe 2
gttcttcagattcatcaacggatatat

SEQ ID NO: 3 - protein, 8 aa, Artificial Sequence
  FLAG peptide sequence
DYKDDDDK

SEQ ID NO: 4 - DNA, 26 bp, Streptococcus suis
  misc_feature: Oligo complimentary to probe 1
ggactcaacaatgttgccgtcaacaa

SEQ ID NO: 5 - DNA, 27 bp, Streptococcus suis
  misc_feature: Oligo complimentary to Probe 2
atatatccgttgatgaatctgaagaac

SEQ ID NO: 6 - protein, 10 aa, Artificial Sequence
  MYC sequence
EQKLISEEDL

(19) United States
(12) Patent Application Publication
(10) Pub. No.: US 2011/0200999 A1
(43) Pub. Date: Aug. 18, 2011

(54) NOVEL DEVICE AND METHOD FOR RAPID DETECTION OF MICROORGANISMS

(76) Inventors: Vishal Soni, Dayton, NJ (US); Yogesh Chander, St. Paul, MN (US)

(21) Appl. No.: 13/027,708

(22) Filed: Feb. 15, 2011

Related U.S. Application Data
(60) Provisional application No. 61/304,982, filed on Feb. 16, 2010.

(51) Int. Cl. C12Q 1/68; C12M 1/34

(52) U.S. Cl. 435/6.11; 435/287.2

(57) ABSTRACT

The present invention is directed to a platform technology for quick and easy detection and identification of single or multiple microorganisms in a sample using peptide labeled oligonucleotides (PLONs). The PLONs are specifically designed to be complementary to certain nucleic acid sequences on a target microorganism. When the PLONs of the present invention are added to nucleic acids extracted from a sample (both biological and/or non-biological), they hybridize to the specific target nucleic acids of the microorganisms, and the PLONs are then detected with one or more specific enzymes coupled to antibodies that are specific to the conjugated peptides attached to the PLONs. The hybridized PLON-enzyme coupled antibody complex is further localized to a test region on a solid matrix or support by the presence of a composition comprising a secondary antibody to enzyme coupled antibody and provides a specific, sensitive, easy to use tool for the detection and identification that does not require any amplification step and any equipment for the final read out. A kit and methods of use of the invention are also provided.

### CROSS-REFERENCE TO RELATED APPLICATIONS

This patent application claims the benefit of U.S. Provisional Patent Application No. 61/304,982, filed Feb. 16, 2010, which is incorporated by reference herein in its entirety.

### FIELD OF THE INVENTION

The present invention relates to a platform technology for detection and identification of microorganisms in a sample. The platform technology of the present invention enables quick, specific and sensitive detection and identification of microorganisms without requiring any amplification technique and without any expensive equipment that is required for other known diagnostic technologies.

### BACKGROUND OF THE INVENTION

Detection and identification of microorganisms including, for example, bacteria, fungi, archea, protists, viruses and prions, is the first step in disease diagnosis. Rapid detection and identification of the disease causing microorganisms is very important, as it not only lowers the treatment costs, but also helps in reducing mortality rates, especially during initial phases of disease outbreak. This was evident during recent outbreaks caused by SARS and novel H1N1 virus (Swine Flu virus), as most of the deaths that occurred during the initial phase of these outbreaks were due to lack of the availability of a rapid identification diagnostic test.

Development of a rapid and easy to use detection system is needed. Microbial culture methods are currently the commonly used methods for the identification of many microorganisms. These culture methods are not classified as rapid, due to the fact that the results of the tests are often not available in less than 24 hours, and usually require 48 to 72 hours to perform. These types of diagnostic methods also require the use of additional laboratory techniques such as staining, microscopy, biochemical tests, etc., to confirm the identity of the microorganism, and also require highly trained professionals to perform them.

There exist some rapid methods for identification of microorganisms. These methods are based on techniques such as the polymerase chain reaction (PCR), real-time PCR (RT-PCR), and microarrays. In PCR and RT-PCR assays, oligonucleotide probes are designed against conserved nucleotide sequences of a target gene for a given pathogen. These probes will specifically hybridize with the genome of microorganism in a sample and then amplify the gene target, thus enabling the detection at very low levels. These molecular biology based assays, a part of molecular diagnostics, are getting popular and gaining market share. Molecular diagnostic assays known in the art are highly specific and sensitive, as they use specific oligonucleotide sequences as probes to target specific genes. However, molecular diagnostics laboratories require expensive equipment and/or technically qualified staff to use these methods, thus increasing the cost of the assay, and decreasing the acceptability of the assay for many existing diagnostic laboratories. It also makes the diagnosis in a field setting, such as in case of an epidemic or pandemic outbreak, very difficult.

Diagnostic kits based on antigen-antibody interaction are also known in the art for the detection of microorganisms. These kits use antibodies specific against specific proteins (antigen) of microorganisms, and are very easy to use, and do not need technically qualified professional or any expensive equipment. However, preparing specific antibodies is a time consuming and expensive process, so rapid development of a test for a new microorganism is not ordinarily possible.

Accordingly, there remains a need for more rapid, specific, inexpensive and easy to use molecular diagnostics in the field of clinical diagnostics. The present invention provides a novel hybridization based molecular approach that allows the design of diagnostic methods, devices and kits that will address this unmet need. These and other advantages of the invention, as well as additional inventive features, will be apparent from the description of the invention provided herein

### BRIEF SUMMARY OF THE INVENTION

The present invention provides a novel technology that integrates oligonucleotide hybridization, antigen-antibody interaction, and colorimetric assay, to develop a specific, inexpensive and easy to use method for rapid identification of microorganisms from variety of samples, including, but not limited to both non-biological (e.g. samples from food, cosmetic, pharmaceutical and laboratories), as well as biological (e.g., nasal swabs, body fluid, feces, blood, and tissue). In addition, the assay and methods of the present invention do not require amplification of nucleic acids in the test sample to detect the presence of the target microorganisms.

In an embodiment, the present invention provides a platform technology and a kit using this technology, for the rapid detection and identification of microorganisms in samples. The platform technology includes peptide labeled oligonucleotides (PLONs) which are specifically designed to be complementary to certain nucleic acid sequences on a target microorganism. While not being limited to any particular mechanism of action, it is contemplated that when the PLONs of the present invention are added to nucleic acids extracted from a sample (both biological and/or non-biological), they will hybridize to the specific target nucleic acids of the microorganisms. Hybridized PLONs are then detected with one or more specific enzymes coupled to antibodies that are specific to the conjugated peptides attached to the PLONs. The hybridized PLON-enzyme coupled antibody complex is further localized to a test region on a solid matrix or support by the presence of a composition comprising a secondary antibody to enzyme coupled antibody. This test region also comprises the substrate for the enzyme, for example, an enzyme such as horseradish peroxidase (HRP).

In an embodiment, the PLON-enzyme coupled antibody complex reacts with the substrate present in the test region of the matrix and produces a colored reaction product. Due to the relatively highly localized concentration of substrate, the reaction produces a highly sensitive and sharp band which is visible to the naked eye.

In another embodiment, the device and methods of the present invention can selectively target and diagnose multiple microorganisms in one reaction. As such, this will increase efficiency and decrease the time required to diagnose specific microorganisms.

### BRIEF DESCRIPTION OF THE SEVERAL VIEWS OF THE DRAWING(S)

FIG. 1 is a schematic of an embodiment of the method of the present invention.

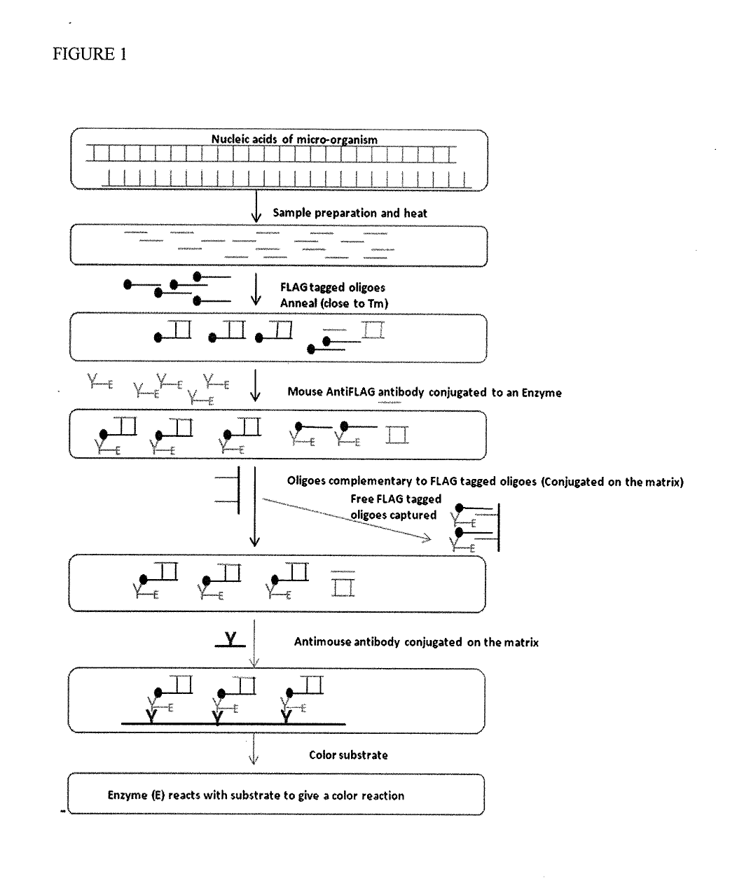

FIG. 2 is a drawing of an embodiment of the device of the present invention.

FIG. 3 is a photograph of an embodiment of the present invention in use, showing three lateral flow devices which are specific for the microorganism Streptococcus suis (S. suis). The left two test devices or test strips have been exposed to test samples having S. suis, and the right test device or test strip is a control where having no exposure to S. suis. The photograph shows that the two test devices have a colorimetric signal in both of their control zones or regions, and in the test zones or regions, indicating a positive test for S. suis. The control device only has a colorimetric signal in the control zone, but no signal in the test zone, indicating that there was no exposure to S. suis, and also that the test device or strip works as designed.

### DETAILED DESCRIPTION OF THE INVENTION

In an embodiment, the present invention provides methods of detection of one or more microorganisms in a sample via oligonucleotide hybridization of a target nucleic acid (DNA or RNA) of one or more microorganisms from a sample (both biological and non-biological) with a peptide labeled oligonucleotide probe (PLON) specific for the target nucleic acid (DNA or RNA) of one or more microorganisms. The PLON comprises an oligonucleotide probe conjugated with a epitope or peptide tag. In an embodiment, for example, the epitope or peptide tag can be a short amino acid sequence, such as, but not limited to, FLAG (DYKDDDDK) (SEQ ID NO: 3), polyhistidine (His), hemagglutinin (HA), myc (EQKLISEEDL) (SEQ ID NO: 6). Other tags which can be used in the present invention comprise Arg-tag, calmodulin-binding peptide, cellulose-binding domain, DsbA, c-myc-tag, glutathione S-transferase, HAT-tag, maltose-binding protein, NusA, S-tag, SBP-tag, Strep-tag, and thioredoxin.

The oligonucleotides comprising the PLONs of the present invention are designed to be complementary to conserved nucleotide sequences of a gene or nucleic acid sequence for a given microorganism. In an embodiment, the labeled oligonucleotide probe of the PLON will specifically hybridize with the nucleic acid target sequence of a microorganism. The labeled hybridized probe will then interact with a first antibody which comprises an antibody conjugated to an enzyme, such as horseradish peroxidase (HRP), and wherein the first antibody is specific for binding the epitope or label on the PLON to labeled probe, creating a first PLON-antibody complex. The PLON is then further concentrated by exposure to a second antibody, which is specific to the first antibody at a specific region of the first antibody, but which does not interfere with the binding of the first antibody to the PLON, or interfere with the binding of the PLON to the target nucleic acid. The bound second antibody to the first PLON-antibody complex comprises the second PLON-antibody complex. This second antibody complex is in proximity to the substrate for the enzyme conjugated to the first antibody, and results in the enzyme reacting with the substrate to form a chromophore. In an embodiment, the reaction of the substrate with the enzyme provides a visual color or signal. In one embodiment, the reaction of the substrate with the enzyme provides a visible line on a test matrix.

In another embodiment, the present invention provides a method of detecting one or more microorganisms in a sample (biological and/or non-biological) comprising: a) obtaining a sample and treating the said sample with a reagent to extract nucleic acids from the sample; b) adding to the nucleic acids from the sample of a), a mixture comprising one or more PLONs specific for a target nucleic acid sequence of one or more microorganisms of interest, wherein each PLON comprises a nucleic acid sequence that is complimentary to that specific target nucleic acid sequence of the one or more microorganisms of interest, and each PLON is also conjugated to an epitope or tag having a specific peptide sequence; c) hybridizing the PLON to the target nucleic acid sequences from the sample of a), to form one or more bound PLON-nucleic acid complexes; d) contacting the bound PLON-nucleic acid complexes of c) with the one or more first binding proteins, wherein the one or more first binding proteins comprise a monoclonal or polyclonal antibody specific for the one or more epitopes or tags on the one or more PLONs, and wherein the first binding proteins are also conjugated to an enzyme; e) binding the one or more first binding proteins to the one or more epitopes or tags on the one or more bound PLON-nucleic acid complexes and remaining unbound PLONs, forming one or more bound PLON-first antibody complexes and/or one or more unbound PLON-antibody complexes; f) contacting the one or more bound PLON-first antibody complexes and/or one or more unbound PLON-antibody complexes of e) with one or more nucleic acid molecules which have nucleotide sequences which are complimentary to the nucleotide sequences of the one or more PLONs, wherein the one or more nucleic acid molecules which are complimentary to the one or more PLONs are bound to a matrix which also contains a chromogenic substrate of the enzyme conjugated to the first binding proteins; g) hybridizing any unbound PLON-first antibody complexes from the sample with the one or more nucleic acid molecules which are complimentary to the one or more unbound PLON-first antibody complexes bound to the matrix, to form a chromophore comprising the control signal; h) contacting the one or more bound PLON-first antibody complexes from g) with one or more second binding proteins comprising an antibody specific for the first antibody, wherein the one or more second binding proteins are bound to the matrix and which also contains a chromogenic substrate of the enzyme conjugated to the first antibody and forming a chromophore comprising the test signal; i) detecting the control and test signal, and determining presence of the one or more microorganisms in the sample.

It is also contemplated that in another embodiment, the present invention provides multiple PLONs comprising target nucleic acid sequences that are specific to different microorganisms so that more than one microorganism could be identified in a single test device or substrate. For example, in an embodiment, the present invention provides a first set of PLONs specific for microorganism A, and conjugated to an epitope such as FLAG, and a second set of PLONs specific for microorganism B, and conjugated to a different epitope such as HA. The test device or matrix of the device comprises a control region and a test region for each of the one or more sets of PLONs to be identified, e.g. a control zone A, and a test zone A, followed by a control zone B and a test zone B, up to control zone N and test zone N. The only limitation would be the size of the substrate and epitopes selected. It is contemplated that there could be multiple different targets in a single test embodiment.

In a further embodiment, the present invention provides a test device or substrate for detecting and identifying one or more microorganisms in a biological sample, the device comprising: a permeable material defining a plurality of portions, including, at least a first portion, a second portion, and a third portion, the portions being positioned so as to permit capillary flow communication with each other, the first portion comprising one or more indicator zones and is also the site for application of the sample, also known as the test cell or sample pad, onto or into the device or substrate, and the first portion also comprising the one or more first binding proteins. The second portion of the test device or substrate comprises one or more control zones and the site for the one or more nucleic acid molecules which are complimentary to the one or more PLONs and further comprises the substrate(s) for the enzyme(s) of the one or more first antibodies to be immobilized therein, the control zone(s) being the site for visually determining the presence of unbound PLON-first binding protein complexes. The third portion(s) of the test device or substrate comprises the test zone, where one or more second binding proteins and enzyme substrate for the enzyme of the PLON-first binding protein-enzyme complex are immobilized therein, wherein the one or more second binding protein specifically binds to the first binding protein and is the site for visually determining the presence of the one or more bound PLON-first binding protein complexes. In a preferred embodiment, the binding proteins are monoclonal antibodies.

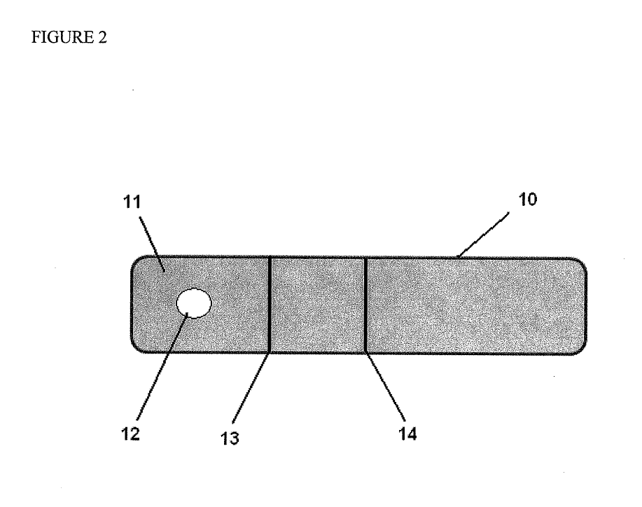

An embodiment of a device of the present invention is provided in FIG. 2. A device or substrate (10) is shown in a strip form. The present invention is not limited to a strip, and could be any shape or orientation that is suitable for a lateral flow type of assay or device. The device (10) is comprised of a substrate (11), which can be a permeable material, such as, for example, nylon. Located proximal to one end is the first portion, which comprises the indicator region of the substrate, and includes a sample well or sample pad (12). Proximal to the first or indicator portion is the second portion, which comprises the control zone (13). Moving further down the device away from the sample pad (12), and proximal to the second portion, is the third portion of the device, which comprises the test zone (14). In an embodiment, a sample will be loaded onto the device (10) with a pipette or other means, and the sample will be allowed to flow via capillary action, in a direction away from the sample pad (12). The sample will flow through the control zone (13) and test zone (14), where it is then visualized by the naked eye, or in an alternative embodiment, through the use of a device, such as, for example, a reflectometer or spectrophotometer or any other device which can be used to detect chemiluminescence. If the control zone displays a chromogenic signal, but the test zone does not, then a determination that the microorganism(s) of interest was not in the sample, is made (i.e., a negative test). If the control zone displays a chromogenic signal, and the test zone displays a chromogenic signal, then a determination that the microorganism(s) of interest was in the sample, is made (a positive test). If the control zone does not display a chromogenic signal, then a determination that a defective test is made (bad control). A bad control could indicate a problem, for example, with the collection step, with the extraction step, or a problem with the PLONs or with the substrate.

In a further embodiment, a method of detecting and identifying one or more microorganisms in a sample (biological and/or non-biological) is provided, the method comprising: (a) providing a permeable material or substrate defining at least a first portion, a second portion, and a third portion, the portions being positioned so as to permit capillary flow communication with each other, the first portion being the indicator zone and upstream from the second and third portions, and the site for application of the liquid sample, and comprising one or more first binding proteins therein, and wherein the permeable material or substrate also having a second portion being the control zone, and comprising one or more nucleic acid molecules which are complimentary to the one or more PLONs, and the second portion also comprising the substrate for the enzyme of the first binding proteins to be immobilized therein, such that the control zone being the site for visually determining the presence of one or more unbound PLON-first antibody complexes, and wherein the permeable material or substrate also having at least a third portion comprising one or more second binding proteins, and also comprising the enzyme substrate for the enzyme of the first binding protein immobilized therein, wherein when the second antibody is capable of specifically binding to the first antibody and is the site for visually determining the presence of the bound PLON-first antibody complexes; (b) applying a sample to the device or substrate at the indicator zone, and (c) detecting the control color at the control zone, and test color at the test zone wherein the accumulation of chromophore produces a color indicative of the presence of a detectable level of one or more microorganisms in the liquid sample. In a preferred embodiment, the at least first and second binding proteins are monoclonal antibodies.

As used herein the term “microorganism” refers to both prokaryotic and eukaryotic microorganisms and includes but not limited to archeabacteria, bacteria, viruses, yeasts, protozoans, inter- and intracellular parasites, such as prions.

As used herein, the term nucleic acid refers to a deoxyribonucleotide (DNA) or ribonucleotide (RNA) polymer in either single stranded (sense or antisense) or double stranded form and may encompass known analogues of natural nucleotides that can function in a similar manner as naturally occurring nucleotides.

As used herein, the term “sample” can be from any hospital, clinic, research laboratory or pharmaceutical, food, environmental, cosmetic, biotech industry. In some embodiments, the sample may be obtained from vertebrate or invertebrate organisms. In certain embodiments, the sample may be a processed or unprocessed nasal or vaginal swabs, body fluids, tissue, organs, feces, urine etc.

The binding proteins of the present invention can include antibodies, and also include recombinant antibodies described herein. As used herein, “recombinant antibody” refers to a recombinant (e.g., genetically engineered) protein comprising at least one of the polypeptides of the invention and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein.

The binding proteins of the invention (including antibodies, and functional portions and functional variants) of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, βphenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

As used herein, the samples will be extracted or otherwise processed to obtain the nucleic acids (e.g., RNA, DNA, etc.) of the target microorganisms by using commonly used sample preparation techniques. In certain embodiments, nucleic acids may be prepared by using commercially available kits from various vendors, for example, such as the DNeasy or RNeasy extraction kits from Qiagen (Valencia, Calif.).

As used herein, the oligonucleotides are designed against unique nucleotide regions of a specific microorganism. More specifically, the oligonucleotides of the present invention are about 12-200 nucleotides long. In some embodiments, more than one oligonucleotide is designed against unique regions. More specifically, in an embodiment, multiple PLONs are used against unique regions of a specific target microorganism. The term “oligonucleotide or PLON” as used herein, refers to any nucleic acid sequence that recognizes and binds to a nucleic acid sequence, such as a RNA or a DNA sequence.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the oligonucleotides of the invention are recombinant. As used herein, the term “recombinant” refers to (i) molecules that are constructed outside living cells by joining natural or synthetic nucleic acid segments to nucleic acid molecules that can replicate in a living cell.

The nucleic acids can be constructed based on chemical synthesis and/or enzymatic ligation reactions using procedures known in the art. See, for example, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N²-carboxypropyl)uracil, and 2,6-diaminopurine. Alternatively, one or more of the nucleic acids of the invention can be purchased from companies, such as Macromolecular Resources (Fort Collins, Colo.) and Synthegen (Houston, Tex.).

The invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand.

As used herein, oligonucleotides of the PLONs of the present invention are synthesized in such a way that they can be conjugated, covalently or non-covalently, to a peptide of about 5-50 amino acids. This technology is commercially available. In certain embodiments, oligonucleotides will be conjugated to some of the commonly available tags such as HA, His, FLAG, Myc, V5 or other peptides tags and variants thereof known in the art.

As used herein, one or more than one of the PLONs are hybridized to the nucleotides in the sample. In certain embodiments, the hybridization is performed at ambient to about 75° C. The duration of the hybridization is about 5 minutes to 24 hours. Preferably, the duration of hybridization is about 10 minutes to 4 hours, or about 15 minutes to 2 hours.

As used herein, the PLONs will hybridize with the samples only if the targeted nucleic acids are present. Hybridized PLONs are further detected with enzyme coupled antibodies that are specific to the peptide portion of PLONs. In certain embodiments, alkaline phosphatase (AP), horseradish peroxidase (HRP) are two examples of the enzymes, but not limited to, that may be used for coupling to the antibodies. In certain embodiments, the amount of enzyme coupled antibody to be used is between about 10 ng/ml-1 mg/ml. Preferably, the amount of enzyme coupled antibody is about 100 ng/ml-0.5 mg/ml or 100 ng/ml-0.25 mg/ml.

The invention further provides an antibody, or antigen binding portion thereof, which specifically binds to epitopes or tags of the PLONs described herein. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the epitopes or tags of the PLONs described herein.

Suitable methods of making antibodies are known in the art. For instance, standard hybridoma methods are described in, e.g., Köhler and Milstein, Eur. J. Immunol., 5:511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). Alternatively, other methods, such as EBV-hybridoma methods (Haskard and Archer, J. Immunol. Methods, 74(2):361-67 (1984), and Roder et al., Methods Enzymol., 121:140-67 (1986)), and bacteriophage vector expression systems (see, e.g., Huse et al., Science, 246:1275-81 (1989)) are known in the art.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235:959-973 (1994).

The invention also provides antigen binding portions of any of the antibodies described herein. The antigen binding portion can be any portion that has at least one antigen binding site, such as Fab, F(ab′)₂, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7:697-704 (1994)). Antibody fragments of the invention, however, are not limited to these exemplary types of antibody fragments.

The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, or can be 100%.

Also, the antibody, or antigen binding portion thereof, are modified to comprise a detectable label, such as, for instance, a radioisotope, a fluorophore (e.g., fluorescein isothiocyanate (FITC), phycoerythrin (PE)), an enzyme (e.g., AP or HRP), and element particles (e.g., gold particles).

As used herein, in an embodiment of the test device of the present invention, the matrix can be comprised of, but not limited to, polyethylene, polystyrene, polypropylene, or a nitrocellulose, nylon, DEAE cellulose membrane.

As described herein, the hybridized PLONs and enzyme coupled antibody will be incubated together. In specific embodiments, they will be incubated together at about 4° C.-50° C.

In a preferred embodiment, the PLONs of the present invention will be incubated together at a temperature of about ambient to about 37° C. The duration of incubation will be about 5-120 minutes. Preferably, the duration of incubation will be about 15-60 minutes.

As described herein, the device and methods of the present invention are very specific and sensitive for detection of one or more microorganisms in a given sample without using amplification technology such as PCR. In certain embodiments, the sensitivity of the methods of the present invention are about 0.1-0.5 fold greater than standard PCR technology. More preferably, the sensitivity of the method of the present invention is about 0.5-2 fold greater than standard PCR technology, and most preferably the specificity of the method of the present invention is 1-5 fold greater than standard PCR technology.

As used herein, the term “variant” includes conservative and/or non-conservative alterations of the peptide sequence of the peptide tags and oligonucleotide sequences. The term “variant” also refers to synthetic equivalents to the native peptide tags or oligonucleotide sequences. In some embodiments, a variant includes one or more amino acid substitutions, insertions, and/or deletions compared to the tag from which it was derived, and yet retains its respective activity.

A “fusion protein,” as used herein, refers to a hybrid protein comprising polypeptide portions derived from two or more different proteins, and is synonymous with “chimeric protein.” In the context of the invention, the first antibody comprises a fusion protein comprising an antibody and an enzyme capable of producing a chromogenic product.

The first binding protein of the present invention can include an antibody which is modified by covalently attaching a moiety to the first antibody. The moiety may be covalently attached to the first antibody, for example, through the use of coupling reagents known in the art, such as those commercially available from, for example, Pierce Chemical Co., Rockford, Ill. The modifying moiety can be any suitable moiety that can be covalently attached to the first antibody. Suitable moieties can be detection molecules. Suitable detection molecules are known to those skilled in the art and include, but are not limited to, enzymes with detectable activities such as HRP, AP, luciferase, beta-galactosidase and beta-glucuronidase, fluorescent moieties, chromophores, haptens and/or epitopes recognized by an antibody. The construction of fusion proteins such as the first or second antibodies of the present invention is routine in the art (see, e.g., U.S. Pat. Nos. 5,130,247 and 6,254,870).

The sample comprising one or more microorganisms of interest can be any suitable sample, but preferably is a sample obtained from a mammal (e.g., a human) or non-mammals (e.g., turkeys). The sample can be a solid sample, such as a tissue sample, or the sample can be fluid, such as a sample of body fluid. For instance, a section of whole tissue can be homogenized to liquefy the components found in the tissue. The tissue sample can be obtained from any suitable organ, including diseased organs (e.g., organs affected by cancer). Suitable fluid samples include, but are not limited to, blood, saliva, serum, plasma, lymph, interstitial fluid, and cerebrospinal fluid.

The first and/or second binding proteins of the present invention can include recombinant antibodies comprising at least one of the inventive antibodies described herein. As used herein, “recombinant antibody” refers to a recombinant (e.g., genetically engineered) protein comprising a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab)₂¹ fragment of an antibody, etc. The polypeptide chain of an antibody, or portion thereof, can exist as a separate polypeptide of the recombinant antibody. The polypeptide of an antibody, or portion thereof, can be a polypeptide of any antibody or any antibody fragment, including any of the antibodies and antibody fragments described herein. In an embodiment the invention provides a test cell for conducting a competitive immunoassay. As disclosed herein, various features of the process and test cell of the invention cooperate to enable untrained personnel reliably to assay a liquid sample for the presence of extremely small quantities of a particular micro-organism while avoiding false positives and simplifying test procedures. The invention is ideal for use in assay test kits which will enable personnel to detect and identify, for example, venereal disease, and other disease, infections, or clinical abnormality, contamination which results in the presence of an nucleic acid marker substance in a sample. The assay process and the cell are engineered specifically to detect the presence of one or more preselected nucleic acid markers present in a samples.

In an embodiment, the assay and/or method of the present invention is conducted by simply placing the test cell of the device or substrate of the present invention in contact with a prepared test sample comprising the PLONs and the nucleic acids extracted from the sample source. The test sample passes through the lateral flow device, and into reactive contact with the one or more test zones (and optionally one or more control zones) visible through a window or windows in the cell's exterior casing. In one embodiment, the PLONs are mixed with the prepared sample and incubated briefly before the test cell is inserted. In another embodiment, the conjugate is disposed in preserved form in the flow path within the cell. If the target nucleic acid molecule is present in the sample, it passes through the inlet and the interior of the cell along the flow path past the test and control sites, where it reacts with immobilized binding protein, e.g., an antibody, at the test site, and perhaps also non-specifically at the control site. A “sandwich” forms at the test site comprising immobilized binding protein-ligand binding protein-colored particle. The presence of the sandwich complex and thus the ligand is indicated by the development of color caused by aggregation of the chromogenic products formed from the enzymatic reaction at the test site. A greater amount of color at the test site than at the negative control site is a positive indication of the presence of the target nucleic acids of the micro-organisms.

In an embodiment, the test device or cell of the present invention can take various forms. It will usually comprise an elongate casing comprising interfitting parts made of polyvinyl chloride, polypropylene, or other thermoplastic resin. Its interior flow path will contain a relatively inert material or a combination of materials suitable for transporting the liquid. In some circumstances it may be preferable to use a material of higher sorptivity as the reservoir, promoting the flow of liquid, and a different material for remaining portions of the flow path.

As used herein, the bound and/or unbound PLONs-antibody-enzyme complex will be loaded onto a solid matrix or substrate or device suitable for lateral flow. In certain embodiments, the matrix will be comprised of Nylon® or its charged derivatives, DEAE-cellulose, nitrocellulose or other matrices commonly used for the art. The construction of the matrix or substrate allows the bound and/or unbound PLONs-antibody-enzyme complex to flow from one end of the substrate or device to the other.

As used herein, the matrix will have at least two regions: A positive control region and a test region. In certain embodiments, multiple test and control regions may be present. In an embodiment, the control region will be closer the sample loading spot. This region will have oligonucleotides that are complementary to the PLONs immobilized to the matrix. The control region will also have enzyme substrate. This region will give a colored reaction due to reaction between unbound or unhybridized (single stranded) PLONs-antibody-enzyme coupled complex with their complementary oligonucleotides. However, hybridized (double stranded) PLONs-enzyme coupled antibody will not react with the test region and move to the test region.

As used herein, the test region will have a secondary antibody specific for the first antibody in the PLONs-antibody-enzyme coupled complex immobilized in a highly localized region. In certain embodiments the area of this localized region is about 0.001-10 mm². Preferably, the area of this localized region is about 0.001-5 mm² or 0.001-1 mm². In certain embodiments, the amount of secondary antibody immobilized in test region is about 10 ng-1 mg.

As used herein, the test region also has substrate for the enzyme that was couple to primary antibody in PLONs-antibody-enzyme complex. This enzyme reacts with its substrate to give a colorimetric reaction. This result of this reaction can be visible through naked eye.

As used herein, in certain embodiments, fluorescent dyes or other methods of art in may be used to detect the hybridized PLONs. This method will however require an equipment, for example, spectrophotometers, fluorimeters or reflectometers, for end point reading.

It is contemplated that in an embodiment of the present invention, the method will be carried out using a lateral flow method, using a gradient lateral flow device (LFD), such as found in U.S. Pat. Nos. 5,710,005 and 6,485,982, incorporated by reference herein. The gradient flow device can have different zones or regions within the chromatographic area.

Indicator Zone. The analyte, in the context of the present invention, being the PLON bound to the target nucleic acid of interest, will move downstream as a front and will next contact the indicator zone, which contains at least one first binding member or protein. In this instance, the binding member or protein is a first antibody and forms the PLON-first antibody complex in the indicator zone, and becomes mobile upon contact with the moving analyte gradient front. The length of the indicator zone can vary, and in a preferred embodiment, will be approximately the same as the length of the analyte gradient front, and the two will be parallel to one another. The indicator zone may also contain an appropriate signaling substance such colored latex beads, or silica, or liposomes that have encapsulated chemiluminescors (e.g., luciferin) or chromophores (e.g., dyes, or pigments).

Because of the properties of the materials within the gradient LFD, the analyte will move through the indicator zone, mobilizing the PLON-first antibody complex therein, and presenting it for subsequent reaction (or non-reaction) with the fixed binding member present in the control zone and in the test zone.

The Control Zone. As used herein, the control zone is a zone that contains a line, or some other configuration, such as a spot, that becomes detectable in a manner that is independent of the analyte gradient and is also included in the device of the present invention. Preferably, the control zone is in the vicinity of the test zone and consists of at least one immobilized second binding member or protein that reacts with some portion of the first binding member or protein, or signaling substance, from the indicator zone. In an embodiment, the control zone comprises a series of oligonucleotides which are complimentary to the one or more target PLONs which were mixed with the DNA in the sample. These complimentary oligonucleotides are bound to the control zone matrix. In another embodiment, the complimentary oligonucleotides are in proximity to the enzyme substrate of the first antibody binding member. As such, the molecules moving into the control zone from the indicator zone are: (1) PLONs bound to target nucleic acid molecules which are now bound to the first antibody binding member; (2) unbound PLONs which are bound to the first antibody binding member; (3) unbound nucleic acid molecules; and (4) unbound first antibody binding member. In the control zone, the complimentary oligonucleotides bind to the unbound PLONs bound to the first antibody binding member, and react with the enzyme on the first antibody binding member to provide a colorimetric signal of a positive control. The control zone also increases the sensitivity of the assay of the present invention by removing the unbound PLONs bound to the first antibody, which would otherwise result in a false positive signal in the test zone.

The test zone and the control zone may be covered with a clear or translucent cover to facilitate visualizing the signal generated, such as in a device of the present invention. The cover may be uniform, or it may be punctuated by, for example, lines or bars that facilitate determining whether the concentration of the analyte falls within a particular range.

The Test Zone. The analyte gradient moves through the indicator zone, and the control zone, as described above, and then downstream to the test zone. The test zone will contain a fixed second binding member that reacts either with the analyte, or with the mobile binding member that has been carried to the test zone from the indicator and control zones. In an embodiment, the test zone comprises an anti-primary antibody (the second antibody) that is bound to the matrix in proximity to the enzyme substrate of the enzyme of the first antibody. When the second antibody comes into contact with, and binds the PLON-first antibody complex, the enzyme conjugated to the first antibody comes into contact with the enzyme substrate, and the detectable test line forms. Other binding members can be used as the fixed second binding member, including, for example, antibody fragments, oligonucleotides or aptamers.

In an embodiment, the first binding member can be conjugated to HRP. The first antibody-HRP enzyme conjugates production is based on the modified method of Nakane (Nakane et al, 1978: Immunoflorescence and Related Staining Techniques, Knapp, et al., eds., p 215-220, Elsivier/North-Holland Biomedical Press, Amsterdam).

Briefly, in an embodiment, HRP is first subjected to an oxidation treatment with sodium m-periodate. This oxidation generates an aldehyde group on the carbohydrate side chain. The antibody and oxidized HRP is then mixed in alkaline pH, allowing the amino group on the antibody to react with the aldehyde group on HRP to form a Schiff base and reduced to a covalent bond between antibody and HRP. The antibody-HRP conjugate is then purified by gel filtration with a sephacryl-300 column. Other methods of preparing HRP conjugates are know in the art and can be used in the context of the present invention.

The second binding protein or antibody of the present invention is an anti-primary antibody such as anti-mouse, anti-rabbit, anti-sheep, anti-goat etc. which are readily available commercially, for example, from Abcam Inc. (Cambridge, Mass.).

### EXAMPLES

The following examples further illustrate the invention but, of course, should not be construed as in any way limiting its scope. This example demonstrates the production of the diagnostic method in accordance with an embodiment of the present invention.

Sample preparation. The biological and/or non-biological samples are treated with a lysis solution for extraction of nucleic acid (DNA or RNA) using a suitable lysis buffer known in the art. For example, a buffer containing 100 mM Tris (pH 8.0), 5 mM EDTA (pH 8.0), 0.2% SDS, 200 mM NaCl, and 100 mg/ml Proteinase K is suitable for an embodiment of the present invention. About 0.5 ml of lysis buffer is then added to the sample and incubated at temperature of between about 25° C. to about 60° C., preferably about 55° C., with rocking or rolling overnight. The sample is removed from incubator and spun for approximately 10 minutes at about 14,000×G. The sample is then transferred to a second tube and 0.5 ml of isopropanol is added and the sample is mixed to extract the nucleic acids. The nucleic acids are transferred to another tube containing a hybridization buffer. Alternatively, kits that are commercially available from vendors, such as Qiagen, may be used.

Hybridization. To the nucleic acids extracted above, labeled oligonucleotide probes (e.g. FLAG tagged oligonucleotide, e.g., PLONs) are added along with the hybridization buffer, for example, 6×SSC, 0.01M EDTA (pH 8.0), 5×Denhardt's solution, 0.5% (w/v) sodium dodecyl sulfate (SDS). The nucleic acids and PLONs are incubated for at least between about 5 to about 30 minutes, preferably about 15 minutes at room temperature, or higher, to facilitate annealing of probe with the target sequences on genomic DNA. This is the sample for loading onto the test matrix or substrate.

The sample is loaded onto the test matrix or substrate with an indicator region having an enzyme, (HRP) conjugated first antibody against the peptide labeled oligonucleotide PLON (e.g. mouse-anti-FLAG antibody-HRP) to create a nucleic acid-PLON-antibody-enzyme complex. Unbound PLON is also bound by the HRP conjugated antibody.

The first antibody located in the indicator zone of the matrix, interacts with the hybridized and free probe and then moves through the matrix and passes into a control zone on the test device or substrate. The control zone comprises a quantity of single stranded oligonucleotides that are complementary to the PLONs, and are bound to the matrix in proximity to the HRP substrate.

Unbound PLONs bound to the first (HRP) conjugated antibody interact with their complimentary oligonucleotides and react with the bound HRP substrate to give a colored line. This line is the positive control line.

The sample continues to move through the test device matrix or substrate to a test zone. Hybridized PLONs bound with the first antibody move into the test zone of the matrix. A test zone may have single or multiple regions. In a test zone, a second antibody against the first antibody is conjugated to the matrix or substrate, e.g. anti-mouse secondary antibody against mouse HRP anti-FLAG.

Hybridized probe interacts with the second antibody and is bound in the test zone. The enzyme reacts with substrate and gives a colorimetric reaction. A colored line in the test zone indicates the presence of nucleic acid from the target microorganism.

In an alternate embodiment, it is contemplated that the method could include multiple oligonucleotide probes of PLONs with different epitopes or tags specific for different microorganisms. The two or more different PLONs can be mixed with the sample and hybridized to the two or more target nucleic acids in the sample. The method can include a test substrate or matrix where there are two or more control zones and two or more test zones in the matrix thus allowing for the ability to test for two or more pathogenic microorganisms in one sample using one test substrate or matrix.

### Example 1

This example demonstrates one embodiment, in accordance with the present invention, for specific detection of Streptococcus suis (S. suis) using PLONs.

Design and synthesis of PLONs: Oligonucleotide probes specific for a conserved region of S. suis is designed by using DNASTAR software (DNASTAR, Inc., Madison, Wis.). Two probes with the following nucleic acid sequences (Probe 1: 5′ TGTTGACGGCAACATTGTTGAGTCC 3′ (SEQ ID NO: 1)), Probe 2: 5′ GTTCTTCAGATTCATCAACGGATATAT 3′ (SEQ ID NO: 2)) are conjugated to DYKDDDDKC (SEQ ID NO: 3) (FLAG tag with an extra cysteine residue) through a SMCC crosslinker (Succinimidyl-4[N-maleimidomethyl]cyclohexane-1-carboxylate) by commercial vendors.

### Example 2

Preparation of samples: A 10% suspension of tissue samples (lung, heart etc.) obtained from diseased pigs infected with S. suis is made in PBS. The suspension is plated on sheep blood agar plates (Becton-Dickinson, Franklin Lakes, N.J.) and incubated in an incubator (with 5% CO2) at 37° C. for about 24 hours. A loopful of culture from the plates is suspended in about 1 ml of PBS in an eppendorf tube for DNA extraction. The tube is vortex mixed to make homogeneous suspension. The sample is then centrifuged at 8000×g and the pellet is reserved. About 1 ml of PBS is then added and the sample is vortexed, and again centrifuged at 8000×g. The pellet is reserved and the supernatant is discarded. The DNA from the cell pellet is extracted using Qiagen DNA extraction kit. The DNA is then further concentrated by ethanol precipitation, and quantified using a spectrophotometer and standard methods.

### Example 3

Preparation of reaction mixture: Approximately 200 ng of S. suis DNA is placed in an eppendorf tube and denatured in a boiling water bath for about 5 minutes, followed by snap cooling an ice bath. The content of the tube are centrifuged to bring the content down. About 20 μM to about 200 μM of PLONs are added followed by addition of anti-FLAG antibody, Tris 10 mM, Tween 20, PCR buffer (1×), and water to make total volume to 50 Content of the tubes are mixed and then centrifuged. The reaction mixture is incubated at 37° C. for about 1 hour. This reaction mixture is ready for loading on the membrane for final detection.

### Example 4

Preparation of Test Matrix or Substrate: in this Embodiment, a Very Thin Strip of commercially available nitrocellulose or nylon membrane approximately 10×1 cm (length×width) is obtained. Two lines are drawn at about 5 cm and 8 cm to indicate a control and a test zone respectively. About 1,000 μmol to about 10,000 μmol of oligonucleotide complimentary to probe region of PLONs (Oligo 1 complimentary to probe 1: 5′-GGACTCAACAATGTTGCCGTCAACAA-3′(SEQ ID NO: 4) and Oligo 2 complimentary to probe 2: 5′-ATATATCCGTTGATGAATCTGAAGAAC-3′ (SEQ ID NO: 5)) are spotted at the control zone ensuring that it covers the entire control zone. The membrane is allowed to air dry, and the oligonucleotides are crosslinked on the membrane with UV light exposure. Anti-mouse secondary antibody is then spotted in the test zone at a concentration as suggested by the manufacturer 1:20 diluted, ensuring that it covers the entire width of the membrane, and allowed to air dry. The membrane is then blocked in 5% Skim milk (prepared in TBS-T), and is then washed twice with 1×TBS buffer and allowed to dry at 37° C. for about 30 minutes. This test matrix or substrate is ready for performing PLONs based detection of S. suis.

### Example 5

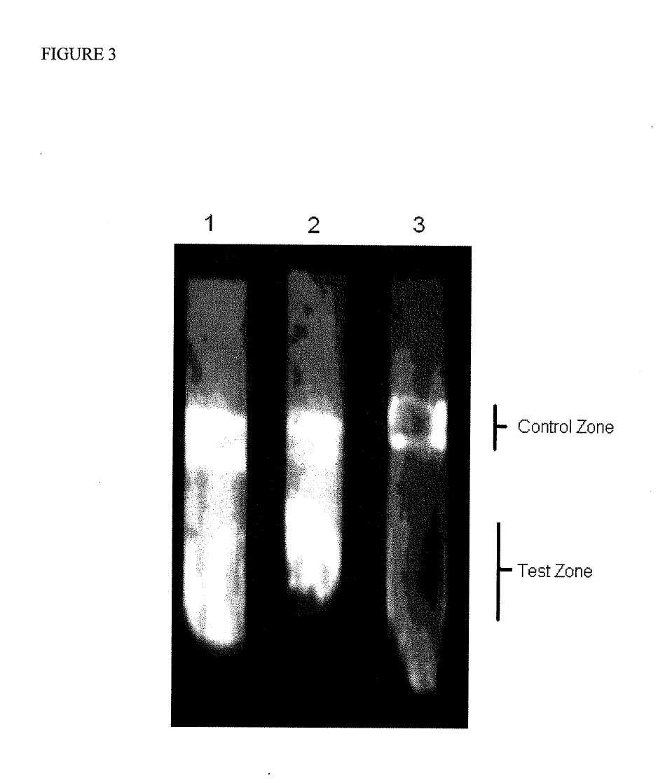

Detection of S. suis using PLONs: The reaction mixture prepared previously in Example 3 is spotted on the sample loading spot or pad, e.g., the indicator zone, on the test matrix or substrate of Example 4. The sample moves and crosses the control and test zones through capillary action. Once entire sample has travelled through the test matrix or substrate (both control region and test region has seen the sample), the membrane is washed twice with 1×TBS buffer, and is treated with HRP reagents. HRP reaction results are visualized by and are recorded by a Gel documentation system GelDoc-It, Ultra-Violet Products (UVP, Upland, Calif.). The results are shown in FIG. 3.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting one or more microorganisms in a sample comprising:
(a) treating the sample with a reagent to release nucleic acids from the sample;
(b) adding to the sample of a) a mixture comprising one or more PLONs specific for a target nucleic acid sequence of one or more microorganisms of interest, wherein each PLON comprises a nucleic acid sequence that is complimentary to a specific nucleic acid sequence of a target microorganism, and each PLON is also conjugated to an epitope or tag having a specific peptide sequence;
c) hybridizing the PLON to the target nucleic acids sequences from the nucleic acids in the sample to form a bound PLON-nucleic acid complex in the sample;
d) contacting the sample of c) with the one or more first antibodies, wherein the one or more first antibodies comprise a monoclonal or polyclonal antibody specific for the one or more epitopes or tags on the one or more PLONs added to the sample in b) and wherein the first antibodies are also conjugated to an enzyme;
e) binding the one or more first antibodies to the one or more epitopes or tags on the one or more bound PLON-nucleic acid complexes and unbound PLONs in the sample in c), forming a bound PLON-first antibody complex and an unbound PLON-antibody complex;
f) contacting the sample of e) with one or more nucleic acid molecules which are complimentary to the one or more PLONs in the sample, wherein the one or more nucleic acid molecules which are complimentary to the one or more PLONs are bound to a matrix which also contains a chromogenic substrate of the enzyme conjugated to the first antibody;
g) hybridizing any unbound PLON-first antibody complexes from the sample with the one or more nucleic acid molecules which are complimentary to the one or more unbound PLON-first antibody complexes bound to the matrix and forming a chromophore comprising the control signal;
h) contacting the sample from g) with a second antibody comprising an antibody specific for the first antibody, wherein the second antibody is bound to the matrix and which also contains a chromogenic substrate of the enzyme conjugated to the first antibody and forming a chromophore comprising the test signal; and
i) detecting the test signal, and determining the presence or absence of the microorganisms of interest in the sample.

2. The method according to claim 1, wherein the matrix is polyethylene, polystyrene, polypropylene, nylon, or a nitrocellulose membrane.

3. The method according to claim 1, wherein said enzyme conjugated to the first antibody comprises an alkaline phosphatase or horseradish peroxidase.

4. The method of claim 1, wherein the sample originated from a biological source.

5. The method of claim 1, wherein the sample originated from a non-biological source.

6. The method according to claim 4, wherein the sample is of plant or animal origin.

7. The method of claim 6, wherein the sample is of animal origin.

8. The method of claim 7, wherein the sample is from blood, urine, feces, tissue, or other bodily fluids.

9. A device for detecting one or more microorganisms in a sample, the device comprising:
a permeable material, matrix or substrate defining a flow path having at least a first portion, a second portion, and a third portion, the portions being positioned so as to permit capillary flow communication with each other, the first portion comprising the indicator zone and the sample pad, and the one or more first binding proteins movably supported therein; the second portion comprising the control zone wherein the one or more nucleic acid molecules which are complimentary to the one or more PLONs and the substrate for the enzyme of the one or more first binding proteins are immobilized therein; the third portion comprising one or more second binding proteins and enzyme substrate for the enzyme of the one or more first binding proteins immobilized therein, wherein the one or more second binding proteins specifically binds to the one or more first binding proteins.

10. The device according to claim 9, wherein the bound or unbound PLON-first antibody complex is transported along the flow path of the substrate by liquid wicking or wetting through the substrate.

11. The device according to claim 9, wherein the matrix is polyethylene, polystyrene, polypropylene, nylon, or a nitrocellulose membrane.

12. The device according to claim 9, wherein said enzyme conjugated to the first binding protein comprises an alkaline phosphatase or horseradish peroxidase.

13. A method for detecting one or more microorganisms in a sample, the method comprising:
(a) providing a permeable material, matrix or substrate defining a flow path having at least a first portion, a second portion, and a third portion, the portions being positioned so as to permit capillary flow communication with each other, the first portion comprising the indicator zone and the sample pad, and the one or more first binding proteins movably supported therein; the second portion comprising the control zone wherein the one or more nucleic acid molecules which are complimentary to the one or more PLONs and the substrate for the enzyme of the one or more first binding proteins are immobilized therein; the third portion comprising one or more second binding proteins and enzyme substrate for the enzyme of the one or more first binding proteins immobilized therein, wherein the one or more second binding proteins specifically binds to the one or more first binding proteins;
(b) applying a liquid sample to the test device at the indicator zone, upstream from the control and test zone so that the sample and the first antibody are transported to the control zone and test zone by liquid wicking or wetting along the flow path; and
(c) observing visually the control result at the control zone, and test result at the test zone wherein the accumulation of chromophore produces a color indicative of the presence of a detectable level of one or more micro-organisms in the liquid sample; and
(d) interpreting the results to determine whether one or more microorganisms are present in the sample.

14. The method for detecting one or more microorganisms of claim 13, wherein the device comprises the device of claim 7.

15. The method of claim 13, wherein the sample originated from a biological source.

16. The method of claim 13, wherein the sample originated from a non-biological source.

17. The method of claim 15, wherein the sample is of plant or animal origin.

18. The method of claim 15, wherein the sample is of animal origin.

19. The method of claim 15, wherein the sample is from blood, urine, feces, tissue, or other bodily fluids.

20. A kit for detecting one or microorganisms is a sample, the kit comprising:
(a) test device comprising a permeable material, matrix or substrate defining a flow path having at least a first portion, a second portion, and a third portion, the portions being positioned so as to permit capillary flow communication with each other, the first portion comprising the indicator zone and the sample pad, and the one or more first binding proteins movably supported therein; the second portion comprising the control zone wherein the one or more nucleic acid molecules which are complimentary to the one or more PLONs and the substrate for the enzyme of the one or more first binding proteins are immobilized therein; the third portion comprising one or more second binding proteins and enzyme substrate for the enzyme of the one or more first binding proteins immobilized therein, wherein the one or more second binding proteins specifically binds to the one or more first binding proteins;
(b) a reagent solution capable of releasing nucleic acid from a sample; and
(c) a container having a mixture comprising one or more PLONs specific for a target nucleic acid sequence of one or more microorganisms of interest, wherein each PLON comprises a nucleic acid sequence that is complimentary to a specific nucleic acid sequence of a target micro-organism, and each PLON is also conjugated to an epitope or tag having a specific peptide sequence.
